(19) United States
(12) Patent Application Publication
(10) Pub. No.: US 2026/0171212 A1
(43) Pub. Date: Jun. 18, 2026

(54) SUSTAINED BEHAVIOR SUPPORT APPARATUS, SUSTAINED BEHAVIOR SUPPORT METHOD AND PROGRAM

(71) Applicant: NTT, Inc., Tokyo (JP)

(72) Inventors: Yasuo ISHIGURE, Tokyo (JP); Tae SATO, Tokyo (JP); Kaori FUJIMURA, Tokyo (JP); Taiga SANO, Tokyo (JP); Asami MIYAJIMA, Tokyo (JP)

(21) Appl. No.: 19/125,023

(22) PCT Filed: Nov. 1, 2022
(86) PCT No.: PCT/JP2022/040925

§ 371 Date: Apr. 28, 2025

(51) Int. Cl. G16H 20/30; A61B 5/00; A61B 5/11; G06Q 10/0639; G06Q 10/10

(52) U.S. Cl. CPC G16H 20/30; A61B 5/1118; A61B 5/681; G06Q 10/06398; G06Q 10/10

(57) ABSTRACT

An activity continuation support device includes: a boredom detection unit configured to detect boredom with certain activity based on a history of an achievement status of a certain person for a goal for the activity; and a goal update unit configured to update the goal based on the history in a case where the boredom is detected, thereby supporting continuation of the activity started to achieve the goal.

### TECHNICAL FIELD

The present invention relates to an activity continuation support device, an activity continuation support method, and a program.

### BACKGROUND ART

As a method of setting a goal for activity and evaluating the degree of achievement, there are various methods in the health field, the learning field, and the like. Even in the health field alone, there is a method of making an evaluation by using various indexes such as the number of steps, blood pressure, blood glucose level, and activity amount.

For example, according to a smartphone application of Non Patent Literature 1, a goal of the number of steps counted by daily walking is set in advance, and a difference from the goal is displayed daily, whereby an increase in motivation to achieve the goal is encouraged.

In addition, the number of times of achievement of the goal in a week or a month is displayed to encourage an increase in motivation to achieve the goal.

Furthermore, in the learning field, online learning services are becoming popular. For example, according to Non Patent Literature 2, a series of lectures by moving images can be taken online, and each individual can take the lectures at any time according to his/her schedule. In this service, attendance statuses and grades can be viewed in a list or a graph, and the degree of achievement can be confirmed.

### CITATION LIST

### Non Patent Literature

Non Patent Literature 1: Diet application FiNC, easy health management with a single application, [online], <URL: https://play.google.com/store/apps/details? id=jp.co.fincapp &hl=jp>

Non Patent Literature 2: gacco—a moving image learning service that you can start immediately and use continuously, [online], <URL: https://gacco.org/>

### SUMMARY OF INVENTION

### Technical Problem

However, according to the method of Non Patent Literature 1, although an achievement status of a goal set by a user himself/herself can be confirmed, it is difficult to determine whether or not the set goal is appropriate because the goal is set by the user himself/herself.

In addition, according to Non Patent Literature 2, although a user is notified of an expiration date of a lecture or the like by e-mail, it is difficult to change setting of a goal according to an achievement status.

The present invention has been made in view of the above points, and an object of the present invention is to provide support for continuation of activity started to achieve a goal.

### Solution to Problem

Therefore, in order to solve the above problems, an activity continuation support device includes: a boredom detection unit configured to detect boredom with certain activity based on a history of an achievement status of a certain person for a goal for the activity; and a goal update unit configured to update the goal based on the history in a case where the boredom is detected.

Advantageous Effects of Invention It is possible to support continuation of activity started to achieve a goal.

### BRIEF DESCRIPTION OF DRAWINGS

FIG. 1 is a diagram illustrating a configuration example of an activity continuation support system according to the first embodiment.

FIG. 2 is a diagram illustrating a hardware configuration example of an activity continuation support device 10 according to the first embodiment.

FIG. 3 is a diagram illustrating a functional configuration example of the activity continuation support device 10 according to the first embodiment.

FIG. 4 is a flowchart for describing an example of a processing procedure executed in the first embodiment.

FIG. 5 is a diagram illustrating the first example of a goal achievement status of a user A in the first embodiment.

FIG. 6 is a diagram illustrating the second example of the goal achievement status of the user A in the first embodiment.

FIG. 7 is a diagram illustrating the third example of the goal achievement status of the user A in the first embodiment.

FIG. 8 is a diagram illustrating an example of information on the number of steps of the user A in the first embodiment.

FIG. 9 is a diagram illustrating a functional configuration example of an activity continuation support device 10 according to the second embodiment.

FIG. 10 is a flowchart for describing an example of a processing procedure executed in the second embodiment.

FIG. 11 is a diagram illustrating a configuration example of a goal information DB 16.

FIG. 12 is a diagram for describing recalculation of an updated goal in a case where boredom of a user A is detected in the second embodiment.

### DESCRIPTION OF EMBODIMENTS

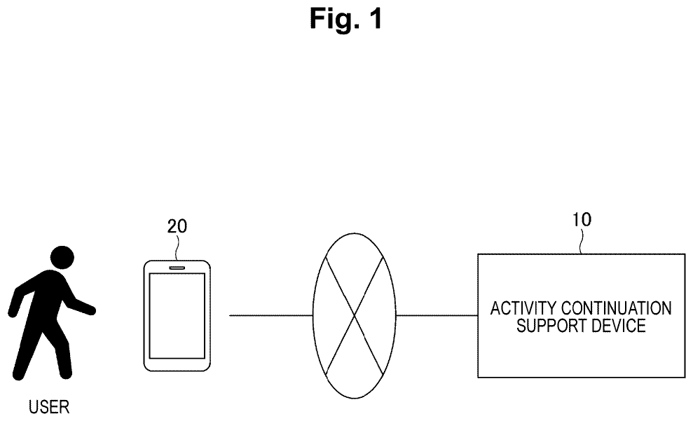

Hereinafter, embodiments of the present invention will be described with reference to the drawings. FIG. 1 is a diagram illustrating a configuration example of an activity continuation support system according to the first embodiment. In FIG. 1, one or more information terminals 20 are connected to an activity continuation support device 10 via a network such as the Internet.

The information terminal 20 is an information processing terminal used by a person who sets a certain goal and starts activity to achieve the goal. For example, a smartphone, a smart watch, or the like may be used as the information terminal 20. For example, walking 10, 000 steps per day may be set as a goal. The information terminal 20 includes a sensor or the like for collecting health-related data such as the number of steps of a user. Depending on the content of a goal, it is also possible to use an application log or the like without using a sensor. Although it is not always necessary to have a communication function as long as functions required in the present embodiment are satisfied, having the communication function makes it possible to implement a more satisfactory application.

The activity continuation support device 10 is one or more computers that execute processing for supporting continuation of activity started by a user to achieve a goal.

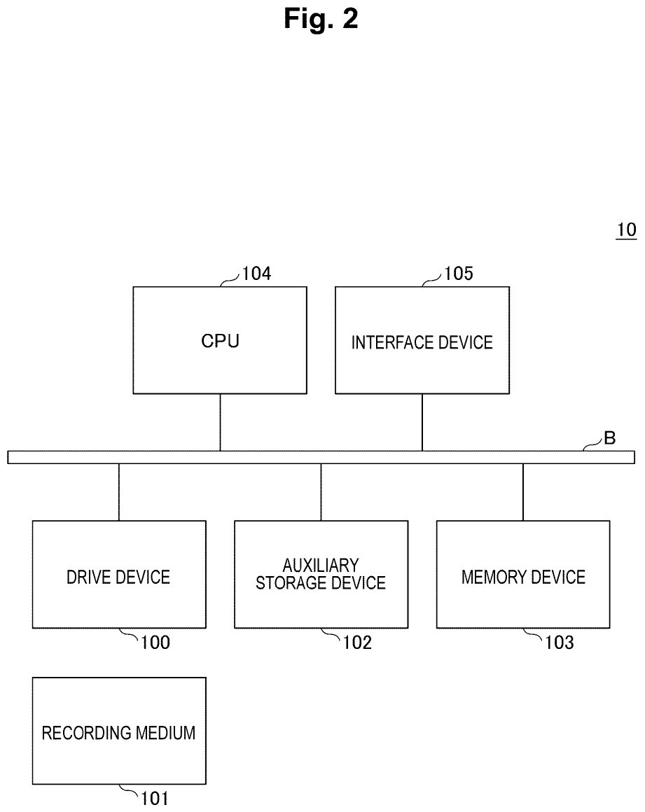

FIG. 2 is a diagram illustrating a hardware configuration example of the activity continuation support device 10 according to the first embodiment. The activity continuation support device 10 of FIG. 2 includes a drive device 100, an auxiliary storage device 102, a memory device 103, a CPU104, an interface device 105, and the like, which are connected to each other by a bus B.

A program for implementing processing in the activity continuation support device 10 is provided by a recording medium 101 such as a CD-ROM. When the recording medium 101 storing the program is set in the drive device 100, the program is installed from the recording medium 101 to the auxiliary storage device 102 via the drive device 100. However, the program is not necessarily installed from the recording medium 101, and may be downloaded from another computer via a network. The auxiliary storage device 102 stores the installed program and also stores necessary files, data, and the like.

When an instruction to start the program is given, the memory device 103 reads the program from the auxiliary storage device 102 and stores the program. The CPU 104 executes a function related to the activity continuation support device 10 according to the program stored in the memory device 103. The interface device 105 is used as an interface for connection to a network.

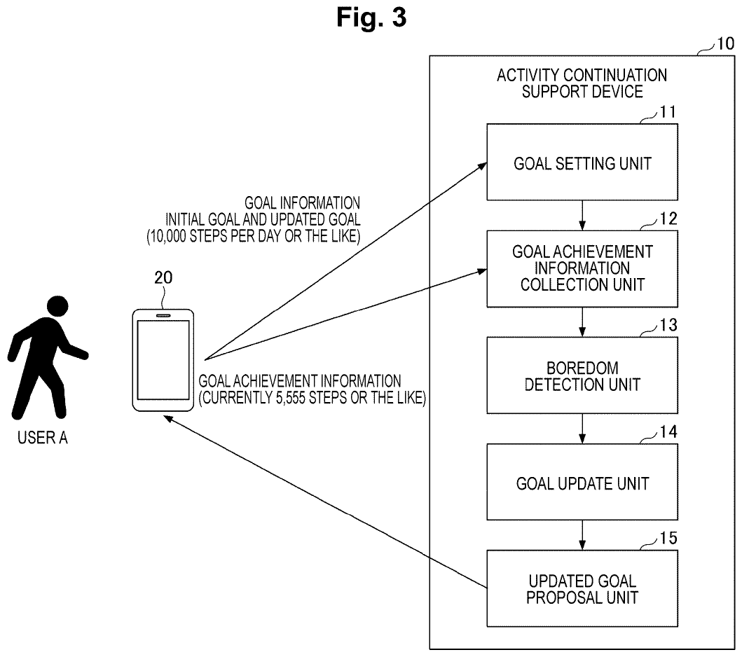

FIG. 3 is a diagram illustrating a functional configuration example of the activity continuation support device 10 according to the first embodiment. In FIG. 3, the activity continuation support device 10 includes a goal setting unit 11, a goal achievement information collection unit 12, a boredom detection unit 13, a goal update unit 14, and an updated goal proposal unit 15.

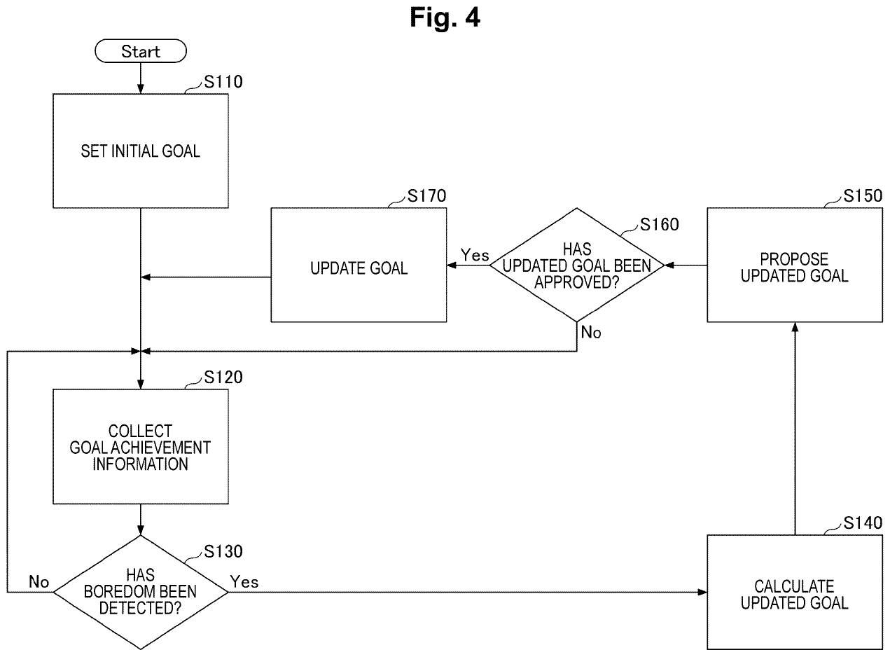

Hereinafter, a processing procedure executed in the activity continuation support system will be described. FIG. 4 is a flowchart for describing an example of the processing procedure executed in the first embodiment.

In step S110, the goal setting unit 11 receives an initial goal set by a user using the information terminal 20. As a basic idea, it is desirable for the user to set a goal from the information terminal 20 from the viewpoint of the user's sense of goal. However, it is not always necessary for the user to set a goal by himself/herself, and in health-related use, for example, a goal may be set by a public health nurse, a registered dietitian, a doctor, or the like in a health guidance interview or the like. Furthermore, in the learning field, a goal may be set by a teacher, a parent, a coach, or the like.

Thereafter, the user performs activity for achieving the goal. In the process, the goal achievement information collection unit 12 automatically collects goal achievement information from the information terminal 20 by previous setting (S120). The goal achievement information is collected every day, for example. Note that the goal achievement information may be any information as long as it is information indicating a goal achievement status (information from which it is possible to determine whether or not the goal has been achieved). The transmission of the goal achievement information from the information terminal 20 to the activity continuation support device 10 is not necessarily automatic. For example, the information terminal 20 may transmit the goal achievement information manually (according to an operation of the user on the information terminal 20).

The boredom detection unit 13 determines the presence or absence of an occurrence of boredom with the activity for achieving the goal on the basis of the collected goal achievement information, for example, at a plurality of predetermined timings (S130). On the basis of not only the goal achievement information for one day but also a history of the goal achievement information in a certain period of time such as one week or one month, the boredom detection unit 13 detects an occurrence of boredom in a case where days on which the goal is not achieved continue (in a case where the days are successive) for a predetermined number of times or more (for example, seven days or more). In the detection of an occurrence of boredom, it is also possible to make an evaluation only with a history of non-achievement of the goal, but in addition, an application log or the like of the information terminal 20 may be referred to. Specifically, the boredom detection unit 13 may determine with higher accuracy whether or not the user is bored with the activity for achieving the goal, assuming that the user is not bored although the goal has not been achieved if an application related to the goal is used although the goal has not been achieved.

If the boredom detection unit 13 detects an occurrence of boredom (Yes in S130), the goal update unit 14 calculates a new goal (updated goal) estimated to be highly likely to be achievable on the basis of the goal information set in the goal setting unit 11 and the history of the goal achievement information collected by the goal achievement information collection unit 12 (S140). In actual use, it is assumed that, in many cases, the goal is updated because the goal has not been achieved. Therefore, a case where the goal has not been achieved will be described.

The goal in a case where the user cannot achieve the goal is the initial goal or the last updated goal. Meanwhile, the goal achievement information from the information terminal 20 (in this case, a history in a certain period of time, such as a daily number of steps of 5,555 steps) is collected by the goal achievement information collection unit 12 and stored in the auxiliary storage device 102 or the like, and thus using the goal achievement information makes it possible to recalculate an updated goal that can be realized. For example, if an average number of steps per day in the past one month is 6, 000 steps and the value is set as an updated goal, the goal is achieved on, for example, about 50% of the days, and it can be expected that the user's self-affirmation feeling is improved.

Subsequently, the updated goal proposal unit 15 performs output for proposing (presenting) the updated goal calculated by the goal update unit 14 to the user via the information terminal 20 (S150). In many cases, the user needs to recognize his/her goal information, and thus the information terminal 20 is caused to present (propose) the updated goal, and transmits the updated goal to the goal setting unit 11 if the user approves the updated goal. In this case (Yes in S160), the goal setting unit 11 updates the goal (S170). On the other hand, in a case where the user himself/herself does not need to be clearly aware of the goal, for example, in a case where the updated goal proposal unit 15 does not consider achievement of the goal itself such as the number of steps but motivates the user by a compliment or the like to the user regardless of his/her achievement of the goal, or in a case where a teacher, a parent, or the like changes the goal on behalf of the user, explicit approval by the user himself/herself may be omitted.

In this way, boredom with the activity for achieving the goal is detected and the updated goal is set, whereby it can be expected that the activity toward the goal will be continued.

Next, the first embodiment will be described on the basis of a specific example. Here, a case will be described where the information terminal 20 is a smartphone (hereinafter, referred to as a “smartphone”), and a certain user (hereinafter, referred to as a “user A”) uses an application (hereinafter, referred to as an “application”) installed in the smartphone, and walking 10,000 steps in a day is set as a goal.

Information on a daily number of steps can be measured by an acceleration sensor of the smartphone, a smart watch capable of cooperating with the smartphone by Bluetooth (registered trademark) or the like, or an activity meter.

When the user A sets information on the number of steps per day (10,000 steps/day) as an initial goal in the smartphone application, the information on the number of steps is registered from the smartphone application to the goal setting unit 11 included in a web application (hereinafter, referred to as a “web application”) of the activity continuation support device 10. The smartphone application transmits daily goal achievement information of the user (today, currently 5,555 steps or the like) to the goal achievement information collection unit 12 included in the web application by use of a communication function included in the smartphone. The boredom detection unit 13 included in the web application periodically (for example, once a day) compares the goal information (10,000 steps/day) with the goal achievement information, and records the achievement status (whether or not the goal has been achieved) on the day.

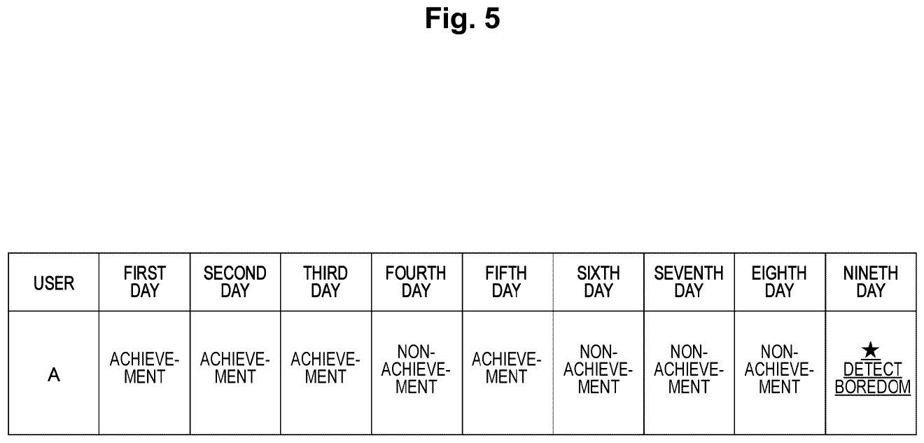

For example, the daily goal achievement status of the user A can be expressed by two types of achievement or non-achievement in the simplest manner as illustrated in FIG. 5. However, as the goal achievement status, achievement or non-achievement may be expressed by a percentage such as 150% or 70%, or may be expressed by the number of steps of the goal (10,000 steps) and the actual number of steps (5,555 steps).

For example, in the case of a condition of detecting that the user is bored when the goal has not been achieved for three consecutive days, the boredom detection unit 13 detects, in the case of FIG. 5, that the user is bored on the eighth day on the ninth day as the next day.

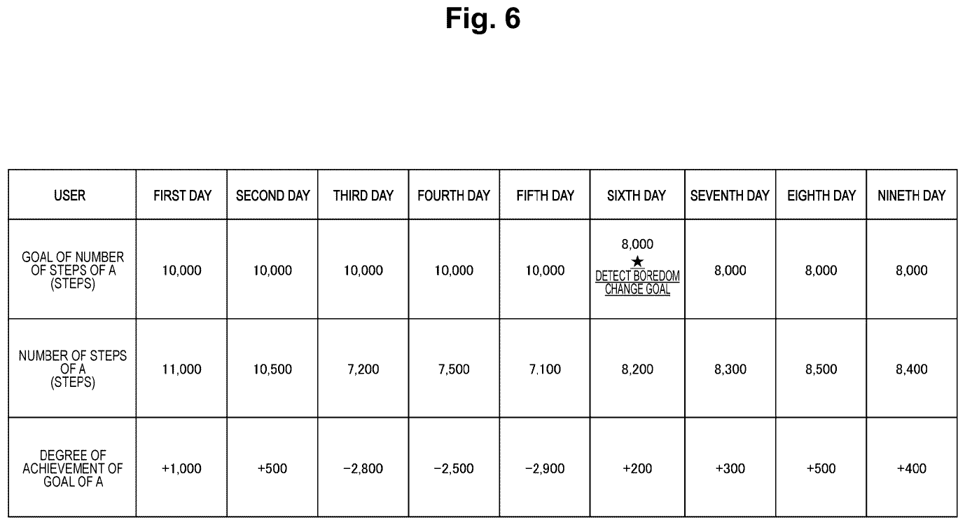

Furthermore, as illustrated in FIG. 6, in a case where the goal achievement status is expressed by a numerical value of the number of steps, the boredom detection unit 13 may compare the goal of the number of steps with an actual measurement value of the daily number of steps for the goal, and evaluate achievement or non-achievement of the goal or the degree thereof.

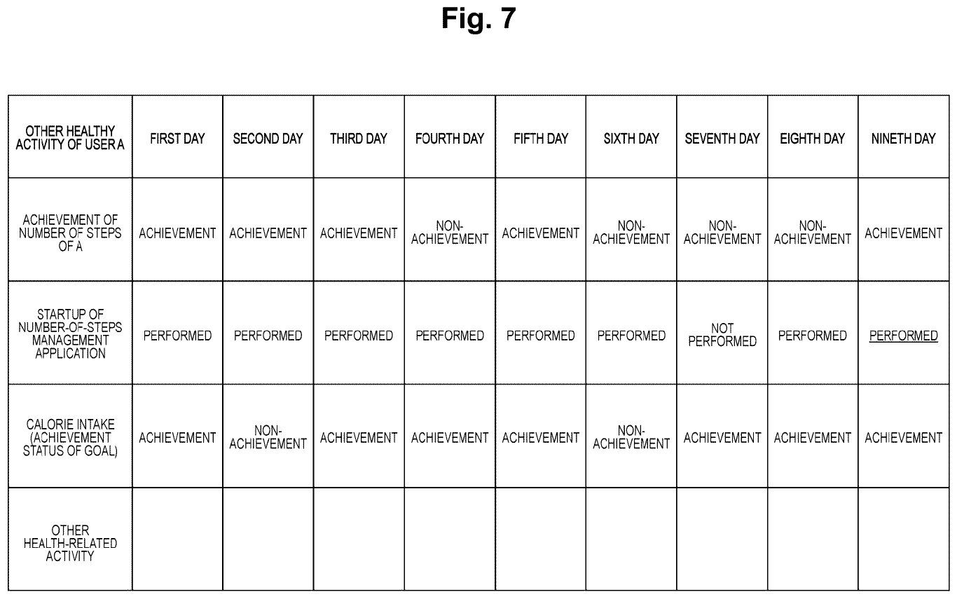

Furthermore, the boredom detection unit 13 may determine the presence or absence of an occurrence of boredom in consideration of other health-related activity, in addition to the healthy activity set as the goal value (the number of steps in this case). As illustrated in FIG. 7, the other health-related activity includes starting a number-of-steps management application to check information on the number of steps on the day, checking the achievement status of a daily goal value of a calorie intake in a case where the goal of the calorie intake is managed together, and the like. By this determination, in a case where the user is not actually bored with walking and the number of steps has not been achieved for some other reason, it is possible to perform advanced boredom detection without erroneously determining that the user is “bored”.

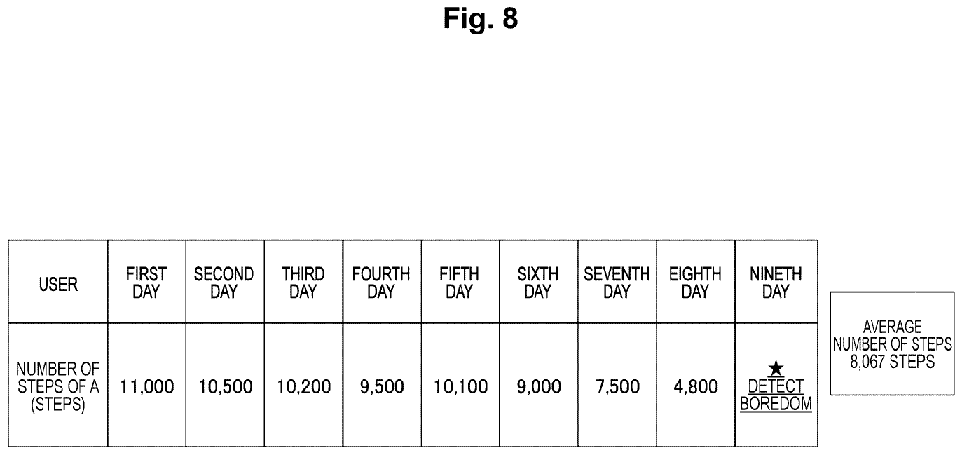

As illustrated in FIG. 8, the goal update unit 14 calculates, for example, an average from a history of past data of the number of steps of the user A (fractions omitted). In this case, the updated goal proposal unit 15 proposes 8,000 steps as a new goal to the user A, so that a more achievable goal can be set. According to this method, a new goal can be presented to the user A, who completely loses sight of the goal, and there is a possibility that activity leading to the goal can be continued.

In addition, for a user who cannot stop the fall of the goal, the past minimum value or the like, such as 3,000 steps per day, is set as a goal, so that it is possible to encourage the user to achieve the minimum goal.

Meanwhile, according to the above method, for a user who continues to achieve a goal for a long time, a higher goal can also be proposed. This makes it possible to change the goal to a more suitable goal for the user who can achieve a higher goal.

The updated goal calculated by the goal update unit 14 is proposed by the updated goal proposal unit 15 to the user through the smartphone application, and the goal is updated with the approval of the user, which can lead to the awareness building of the user. In addition, even in a case where the user himself/herself does not approve lowering the goal, the user is aware of the high goal again, which can lead to more positive activity.

In this way, detecting boredom and updating the goal can lead to promotion of continuation of the user's activity.

As described above, according to the first embodiment, the degree of achievement of a goal is evaluated within a certain period of time, and an achievable goal is proposed and set, so that it is possible to support continuation of activity started to achieve the goal. That is, daily achievement of a goal that is difficult to continue is facilitated, and the self-affirmation feeling is enhanced, which can enhance the possibility that the activity is continued.

Next, the second embodiment will be described. In the second embodiment, points different from the first embodiment will be described. Points not specifically mentioned in the second embodiment may be the similar to those in the first embodiment.

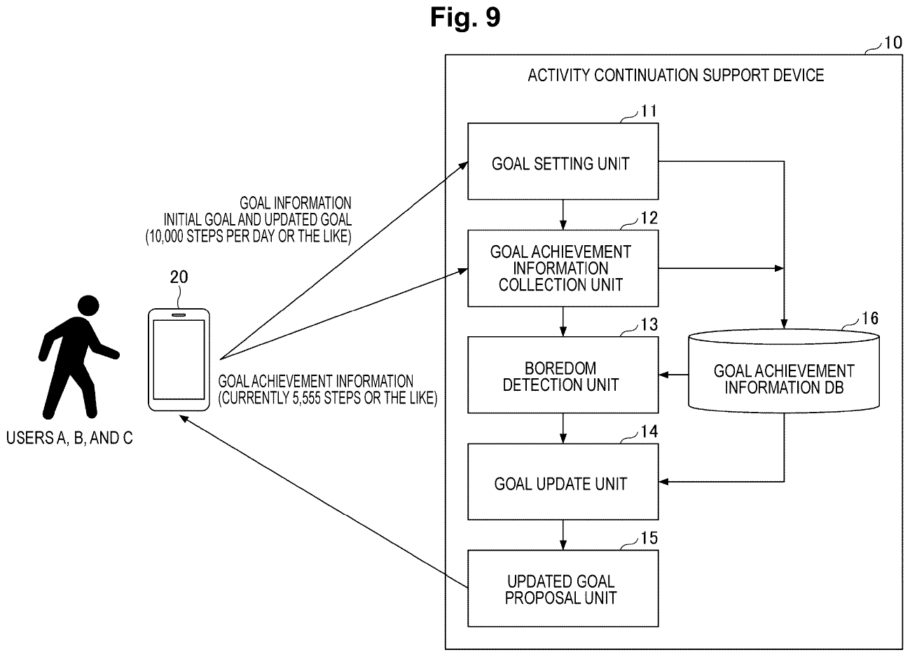

FIG. 9 is a diagram illustrating a functional configuration example of an activity continuation support device 10 according to the second embodiment. In FIG. 9, the same or corresponding parts as those in FIG. 3 are denoted by the same reference numerals, and the description thereof will be omitted as appropriate.

In FIG. 9, the activity continuation support device 10 further includes a goal information DB 16. The goal information DB 16 can be implemented by use of, for example, an auxiliary storage device 102, a storage device connectable to the activity continuation support device 10 via a network, or the like.

The goal information DB 16 is a database (storage unit) that stores an initial goal, goal achievement information, a boredom detection result, an updated goal, and the like for a plurality of users (users A, B, C, and the like in the example of FIG. 9).

In the second embodiment, in a case where boredom of a certain user is detected, a goal update unit 14 calculates an updated goal of the certain user using information regarding another user stored in the goal information DB 16.

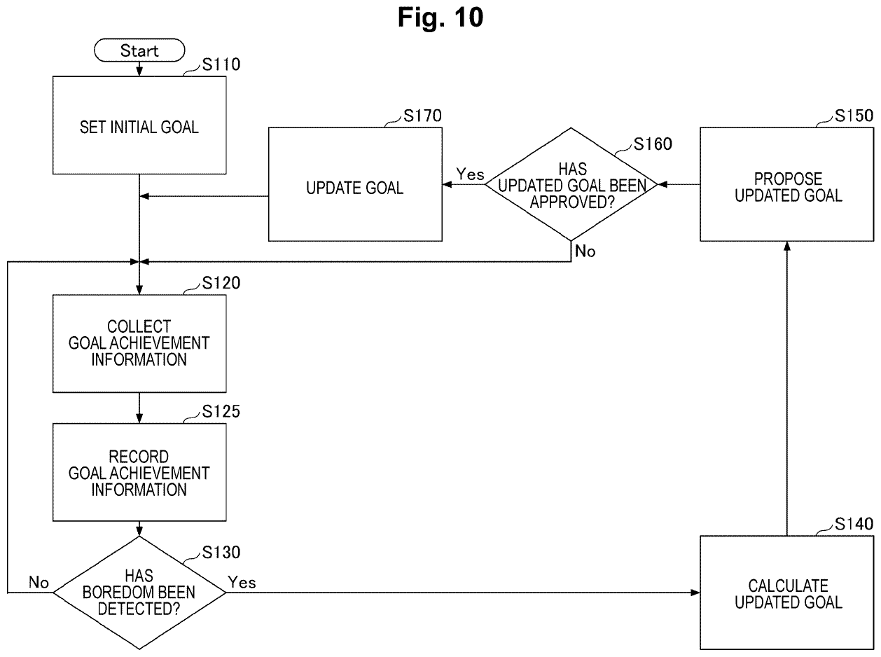

FIG. 10 is a flowchart for describing an example of a processing procedure executed in the second embodiment. In FIG. 10, the same steps as those in FIG. 4 are denoted by the same step numbers, and the description thereof will be omitted. In FIG. 10, step S125 is added after step S120. In addition, step S140 is replaced with step S140a.

In step S125, a goal achievement information collection unit 12 records collected goal achievement information in a goal achievement information DB.

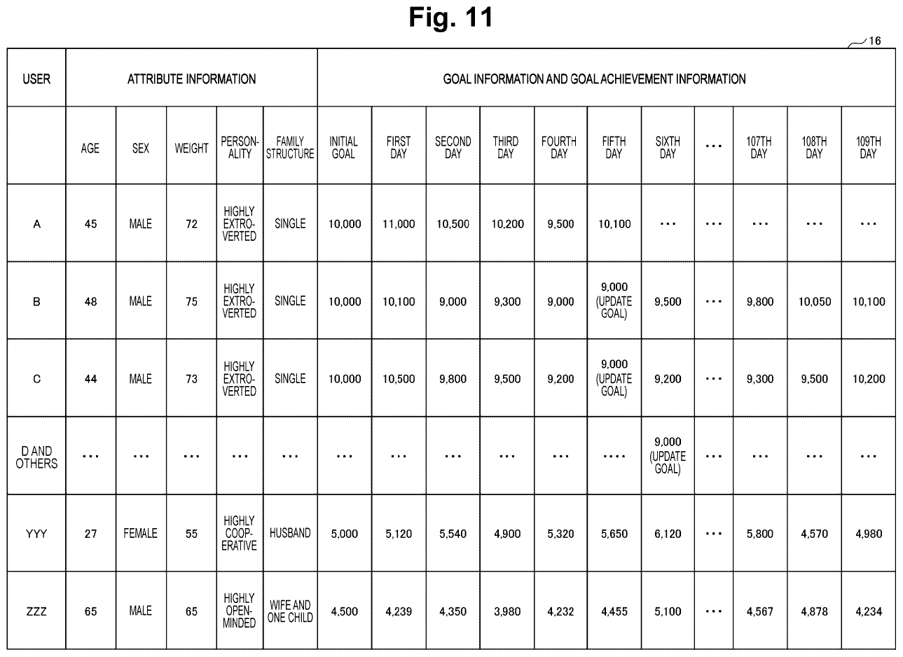

FIG. 11 is a diagram illustrating a configuration example of the goal achievement information DB. As illustrated in FIG. 11, the goal achievement information DB is a database that can store items such as attributes, goal information, and goal achievement information for each of a plurality of users.

The attributes are, for example, items indicating physiological characteristics such as age, sex, and weight, personality characteristics, social characteristics such as a family structure, and the like.

The goal information and the goal achievement information are items including an initial goal, daily goal achievement information, and an updated goal in a case where the goal is updated.

The goal achievement information collection unit 12 records goal achievement information in a column corresponding to a day in a record corresponding to a user from which the goal achievement information has been collected.

In step S140a, the goal update unit 14 recalculates an updated goal of a user detected to be bored on the basis of a goal of a user whose goal achievement continues for a certain period of time or more (for example, for a number of days greater than or equal to a preset threshold) (hereinafter, such a user is referred to as a “reference user”) among one or more other users having attributes (physiological characteristics such as age, sex, and weight, personality characteristics, and social characteristics such as a family structure, and the like) similar to those of the user detected to be bored in a predetermined standard. Regarding the similarity of the attributes, for example, the attributes may be expressed by numerical vectors and evaluated on the basis of the similarity between the vectors. At this time, the correspondence between the value of each of the items constituting the attributes and a numerical value may be determined in advance. Alternatively, the similarity of the attributes may be evaluated on the basis of another standard (method).

Furthermore, for the user detected to be bored, recalculation of the updated goal based on the goal of the reference user may be performed by calculation of an average of latest goals of reference users.

Note that the user as a reference user may be limited to a user whose initial goal is the same as that of the user detected to be bored (or who has an initial goal whose difference from that of the user detected to be bored is smaller than a threshold). With this limitation, it is possible to reduce the possibility that the updated goal of the user detected to be bored deviates from the initial goal due to the influence of a goal of a user whose initial goal deviates.

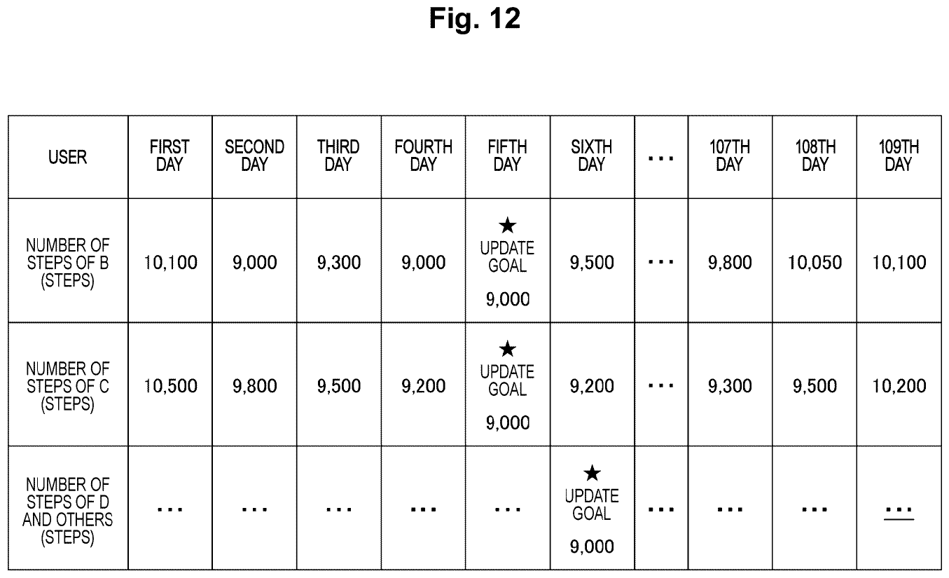

FIG. 12 is a diagram for describing recalculation of an updated goal in a case where boredom of the user A is detected in the second embodiment.

FIG. 12 illustrates a history of goal information and goal achievement information of each user having attributes similar to those of the user A. According to FIG. 12, a user B and a user C are detected to be bored on the fourth day, and an updated goal of 9,000 steps is set on the fifth day. Even for the other users, the updated goal of 9,000 steps is set on the sixth day. Assume that, thereafter, these users have achieved the updated goal (or no boredom has been detected) until the 109th day on which boredom of the user A is detected. In this case, the goal update unit 14 calculates 9,000 steps, which correspond to an average of the updated goals of these users, as an updated goal of the user A.

As a result, it is possible to set a more effective goal for continuation of goal achievement activity.

Note that, as in the first embodiment, for the detection of boredom, a smartphone operation log such as information on startup of a number-of-steps management application, information on a calorie intake (an achievement status of a goal), and other health management information (vital data such as body weight, blood pressure, and blood glucose level) as illustrated in FIG. 7 may be used.

Although the embodiments of the present invention have been described in detail above, the present invention is not limited to such specific embodiments, and various modifications and changes can be made within the scope of the gist of the present invention described in the claims.

### REFERENCE SIGNS LIST

10 Activity continuation support device
11 Goal setting unit
12 Goal achievement information collection unit
13 Boredom detection unit
14 Goal update unit
15 Updated goal proposal unit
16 Goal information DB
20 Information terminal
100 Drive device
101 Recording medium
102 Auxiliary storage device
103 Memory device
104 CPU
105 Interface device
B Bus

1. An activity continuation support device comprising:
a memory; and
a processor coupled to the memory and configured to:
detect boredom with certain activity based on a history of an achievement status of a certain person for a goal for the activity; and
update the goal based on the history in a case where the boredom is detected.

2. The activity continuation support device according to claim 1, wherein
the processor is configured to detect the boredom in a case where non-achievement of the goal in the history continues for a predetermined number of times or more.

3. An activity continuation support device comprising:
a memory; and
a processor coupled to the memory and configured to:
detect boredom with certain activity based on a history of an achievement status of a certain person for a goal for the activity; and
update the goal based on a goal achieved by another person different from the certain person in a case where the boredom is detected.

4. The activity continuation support device according to claim 3, wherein the another person is a person having an attribute similar to an attribute of the certain person in a predetermined standard.

5. The activity continuation support device according to claim 1, wherein
the processor is further configured to perform output for proposing a goal updated.

6. An activity continuation support method, wherein a computer executes:
of detecting boredom with certain activity based on a history of an achievement status of a certain person for a goal for the activity; and
updating the goal based on the history in a case where the boredom is detected.

7. (canceled)

8. A non-transitory computer-readable recording medium storing aprogram for causing a computer to execute the activity continuation support method of claim 6.
